(19) United States
(12) Patent Application Publication
(10) Pub. No.: US 2013/0334084 A1
(43) Pub. Date: Dec. 19, 2013

(54) CONTUSION TAPE

(71) Applicant: Ray Arbesman, Toronto (CA)

(72) Inventor: Ray Arbesman, Toronto (CA)

(21) Appl. No.: 13/902,313

(22) Filed: May 24, 2013

(30) Foreign Application Priority Data
May 24, 2012 (CA) 2777975

(51) Int. Cl. A61F 13/02; A61F 15/00

(52) U.S. Cl. CPC A61F 13/025; A61F 15/002
USPC 206/441; 602/55

(57) ABSTRACT

A contusion patch comprising a high stretch therapeutic tape with an adhesive backing is disclosed in this specification. The tape has an anchoring portion that adheres to a contusion and a plurality of fingers that extend from the anchoring portion. The tape is less than about 5 inches in length and about 2 inches in width when unstretched. A frangible release liner covers the adhesive backing of the tape prior to application. The release liner has a greater surface area than the tape, so that an exposed strip of the release liner extends around the tapes perimeter. The release liner is scored at junctions between the anchoring portion and the fingers to allow selective removal during staggered application of the tape.

### CROSS-REFERENCE TO RELATED APPLICATIONS

This application claims benefit of Canadian Patent Application No. 2,777,975, filed May 24, 2012, which is herein incorporated by reference in its entirety.

### BACKGROUND OF THE INVENTION

1. Field of the Invention

The invention relates to articles for use in the treatment of minor soft tissue injuries, and more particularly to an adhesive contusion patch for use in the treatment of contusions and minor skin abrasions.

2. Description of the Related Art

A relatively new tool in the physiotherapy and sports medicine arsenal is high-stretch adhesive support tape. Such tape is distinct from traditional “sports” tape which is used to isolate and restrain a body part to protect it and allow healing. It is also distinct from traditional Tensor®-type wrap bandages, which although stretchable, are primarily used for bracing an injury. High-stretch adhesive support tape, by contrast, is used for the purpose of positioning a body part (typically a joint) while permitting a high degree of natural mobility and is made from a high-stretch woven fabric matrix with a strong adhesive backing.

High-stretch adhesive support tape is used by physiotherapists and other sports medicine practitioners to support joints, muscles, tendons, ligaments and other parts of the body. The tape is generally used to assist weak musculature by placing a mild degree of tension across the supported body part, in effect acting as an auxiliary muscle. The tension applied by the tape is generally controlled by regulating the amount the tape is stretched during application.

The tape adhesive is strong enough that opposite ends of a length of tape applied to the body will remain adhered even when the tape length is under tension and the body part is in regular active use. This contrasts with other tapes that are either non-adhesive or adhesive only to themselves. These tapes can be wrapped repeatedly on a body part or dispensed from a self-adhesive roll but cannot be adhered directly to the body.

One particularly effective type of high-stretch adhesive support tape is Kinesio Tex™ by Kinesio Co., Ltd. of Japan. This tape has many of the advantageous properties discussed above. However, it suffers several drawbacks, relating to the fact that it is sold in rolls, which must be customized for application according to the imagination and skill of the doctor. A physiotherapist or sports doctor must cut off a section of the tape from a roll, cut the length of tape further into a therapeutic shape, and apply it to the patient. However, cutting the tape inadvertently compromises the tapes underlying fabric weave, creating loose ends in the thread matrix that cause the tape to fray and lift from the skin. Additionally, the cutting, shaping and positioning (tensioning) process are left to the complete discretion of the doctor. It is a trial and error process. The doctor is effectively trying to estimate a position, length, shape and degree of tension that will cause a subjective improvement in sensation, range of motion or pain relief in the patient.

Some manufacturers have introduced precut lengths of tape in several simple shapes to address fraying concerns. These manufacturers also provide instructions for applying the tape to various parts of the body. However, the application process (positioning and tensioning the tape) has not changed and the instructions are often complicated, requiring several precut lengths to be applied in various orientations and tensions. The tape may also still need to be cut before it can be applied in some circumstances, negating the advantage of precut lengths.

Pre-shaped support articles are a recent development that addresses several deficiencies of roll tape and simple precut tape lengths. Pre-shaped support articles are cut into body part specific shapes prior to sale and are often packaged with instructions for their application. Some examples of precut support articles are disclosed in U.S. patent application Ser. No. 12/526,829 (published as US 2010/0016771).

SpiderTech® precut supports are one particularly effective type of shape tailored high stretch adhesive support tape. Each precut SpiderTech® support article has an specific shape that is targeted at a particular body part. Such shapes may be quite elaborate. Some of the highly customized kinesiology tape supports produced by SpiderTech® are illustrated in the following US design patents: USD 608,896—shoulder tape; USD 616,554—knee tape; USD 625,828—neck tape; USD 616,553—lower back tape; USD 335,718—calf and lower leg tape; USD 613,415—foot arch tape; USD 608,893—wrist tape.

Pre-shaped kinesiology tape supports are often accompanied by comprehensive instructions (either included in the support packaging or online) that explain how the tape is to be applied. The instructions typically specify how each section of the tape should be positioned on a specific body part and the amount of tension (stretch) that should be applied to each section. Packaged instructions typically have several diagrams that step the user through the application process. Web based demonstrational videos are also used by some manufacturers and the tape packaging may direct a user to the manufacturer's website for guidance. Demonstrational videos are typically presented by a physician or other health professional who applies the manufacturer's tape to a model in order to exhibit the correct application procedure.

Despite these advances in kinesiology tape technology and education, effective application of the tape is still complicated and may be beyond the capabilities of most end users. In fact, many manufacturers offer educational courses to teach physiotherapists and other health professionals how to apply their particular form of tape.

Further, none of these products is particularly suitable for small and very localized minor injuries (e.g. localized contusions or bruises).

### SUMMARY OF THE INVENTION

It would be desirable to provide a simple patch of versatile precut kinesiology tape that can be used on any small area of the body. For example, it is believed that such a patch could be particularly useful in the treatment of contusions (bruises) or other localized minor injuries or swelling. According to a first aspect of the invention, a contusion patch kit is provided. The kit includes a high stretch therapeutic tape with an adhesive backing (the tape having an anchoring portion that adheres to a contusion and a plurality of fingers that extend from the anchoring portion), a frangible release liner covering the adhesive backing of the tape, and instructions directing a user to apply the contusion patch to a nonspecific part of the user's body using any orientation of the fingers in the user's discretion. The release liner has a greater surface area than the tape so that an exposed strip of the release liner extends around the tape's perimeter. Further, the release liner is scored at junctions between the anchoring portion and the fingers to allow selective removal of the release liner during staggered application of the tape.

The therapeutic tape of the contusion patch is preferably less than about 5 inches in length and about 2 inches in width when unstretched. Ideally the contusion patch is made from therapeutic tape with a stretch ratio that corresponds to the elasticity of human skin. The stretch ratio of the therapeutic tape may be about 1.4 or greater.

The release liner may be marked with visual indicators to direct application of the patch. Specifically, the release liner may be marked with numerals that indicate the order in which sections of the patch are to be applied.

In one embodiment, the tape preferably has a central anchoring portion and four fingers that extend from the anchoring portion, the fingers being arranged in pairs that extend from opposing sides of the anchoring portion to allow the tape to be applied in an ‘X’ configuration.

In another embodiment, the tape preferably has a central anchoring portion and two fingers that extend from one side of the anchoring portion to allow the tape to be applied in a ‘Y’ configuration.

The contusion patch may be supplied in a kit with instructions for applying the patch to a contusion. Ideally, the instructions direct a user to:

i. separate the release liner along the scored junction between the anchoring portion and the fingers,

ii. remove the release liner from the anchoring portion of the tape to expose the adhesive backing,

iii. apply the exposed adhesive backing of the anchoring portion to a contusion so that the tape adheres to the user's skin, and

iv. remove the release liner from each of the fingers in turn and apply the exposed adhesive backing to the user's skin.

The instructions preferably direct the user to stretch each finger after removing the release liner and secure the respective finger in place while stretched by pressing the exposed adhesive backing against the user's skin. Preferably the instructions also direct the user to secure the anchoring portion of the tape to the user's skin without any tension in the anchoring portion.

The kit may include a plurality of contusion patches. Ideally the individual patches would be connected end-to-end, with each patch being folded flat and successive patches resting on top of a previous patches. Preferably adjacent patches would be connected by the frangible release liner. The frangible release liner may be scored between adjacent patches to allow the individual patches to be easily separated.

The therapeutic tape may be coated with a dermatologically-acceptable acrylic adhesive to create the adhesive backing.

According to a second aspect of the invention, a contusion patch kit is provided including a high stretch therapeutic tape with an adhesive backing, a frangible release liner covering the adhesive backing of the tape, and instructions for applying the contusion patch to a nonspecific part of the user's body using any orientation of the fingers in the user's discretion. The tape is functionally divided into sections including an anchoring portion that adheres to a contusion and a plurality of fingers that extend from the anchoring portion. The release liner has a greater surface area than the tape so that an exposed strip of the release liner extends around the tape's perimeter. Further, the release liner is scored at junctions between adjacent sections of the tape to allow selective removal of the release liner during staggered application of the tape.

The release liner is marked with indicators that dictate the order in which each section of the tape is to be applied. The instructions reference the release liner indicators and direct a user to:

i. separate the release liner along the scored junction between adjacent sections of the tape,

ii. remove the release liner from a particular section of the tape indentified by one of the indicators to expose the adhesive backing of that section,

iii. apply the exposed adhesive backing of the section to a contusion so that the tape adheres to the user's skin, and

iv. remove the release liner from each of the other sections in an order dictated by the indicators marked on the release liner in turn and apply the exposed adhesive backing to the user's skin.

The instructions preferably direct the user to stretch each finger after removing the release liner and secure the respective finger in place while stretched by pressing the exposed adhesive backing against the user's skin. Preferably the instructions also direct the user to secure the anchoring portion of the tape to the user's skin without any tension in the anchoring portion.

The release liner preferably has a greater surface area than the tape so that an exposed strip of the release liner extends around the tapes perimeter to reduce accidental exposure of the adhesive backing (and prevent fraying of the edges prior to use).

The therapeutic tape of the contusion patch is preferably less than about 5 inches in length and about 2 inches in width when unstretched. Ideally, the contusion patch is made from therapeutic tape with a stretch ratio that corresponds to the elasticity of human skin. The stretch ratio of the therapeutic tape may be about 1.4 or greater.

The release liner may be marked with visual indicators to direct application of the patch. Specifically, the release liner may be marked with numerals that indicate the order in which sections of the patch are to be applied.

In one embodiment, the tape preferably has a central anchoring portion and four fingers that extend from the anchoring portion, the fingers being arranged in pairs that extend from opposing sides of the anchoring portion to allow the tape to be applied in an ‘X’ configuration.

In another embodiment, the tape preferably has a central anchoring portion and two fingers that extend from one side of the anchoring portion to allow the tape to be applied in a ‘Y’ configuration.

The kit may include a plurality of contusion patches. The individual patches may be connected end-to-end, with each patch being folded flat and successive patches resting on top of a previous patches. Preferably adjacent patches would be connected by the frangible release liner. The frangible release liner may be scored between adjacent patches to allow the individual patches to be easily separated.

The therapeutic tape may be coated with a dermatologically-acceptable acrylic adhesive to create the adhesive backing.

The contusion patches disclosed in this specification are defined in some circumstances by their ‘stretch ratio’. For the purpose of this specification, the ‘stretch ratio’ represents the percentage elongation of the underlying tape under tension and is a measure of the maximum or final stretched length divided by the initial unstretched length (λ=I/L).

### BRIEF DESCRIPTION OF THE DRAWINGS

So that the manner in which the above recited features of the invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a top elevation of an ‘X’ configuration contusion patch after application to a contusion.

FIG. 2 is a top elevation of the contusion patch illustrated in FIG. 1 prior to application with a removable release liner covering the adhesive backing.

FIG. 3 is a bottom elevation of the contusion patch illustrated in FIG. 1 depicting numerical indications printed on the various sections of the release liner and scored delimitations between adjacent sections of the contusion patch.

FIG. 4 is a top elevation of an ‘Y’ configuration contusion patch after application to a contusion.

FIG. 5 is a top elevation of the contusion patch illustrated in FIG. 4 prior to application with a removable release liner covering the adhesive backing.

FIG. 6 is a bottom elevation of the contusion patch illustrated in FIG. 4 depicting numerical indications printed on the various sections of the release liner and scored delimitations between adjacent sections of the contusion patch.

FIG. 7 is a top schematic representation of the contusion patch of FIG. 4 demonstrating the stretch ratio of the therapeutic tape. The anchoring portion and one of the fingers have been applied to a subject while another finger is unstretched and is still attached to the release liner.

FIG. 8 is a side elevation of a plurality of contusion patches connected end-to-end and stacked flat in a pile.

FIG. 9 is a flow chart representation of a method for applying a contusion patch.

### DETAILED DESCRIPTION

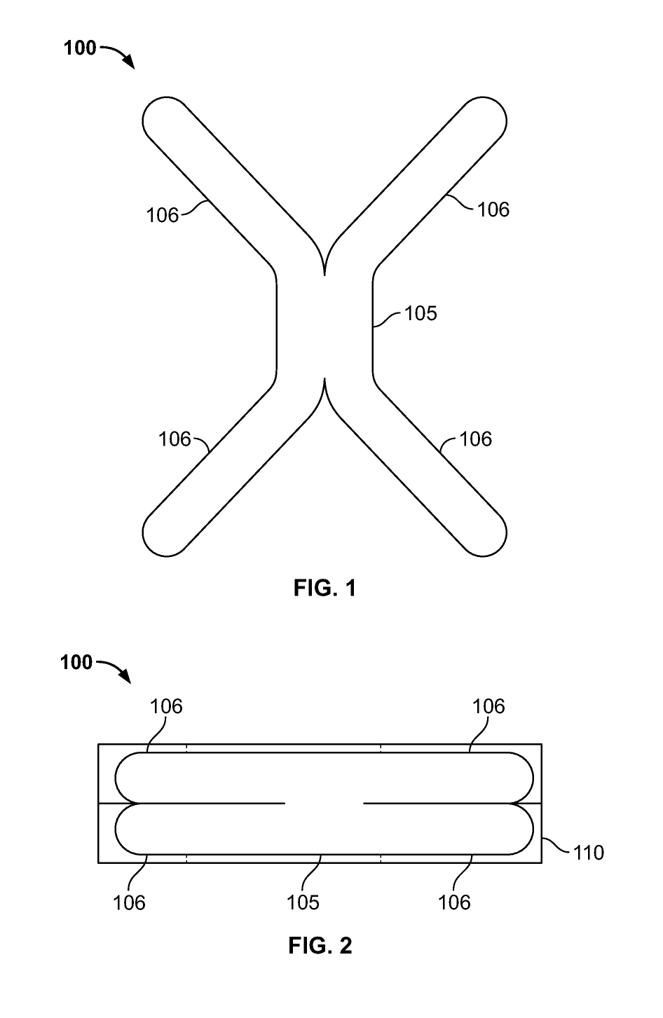

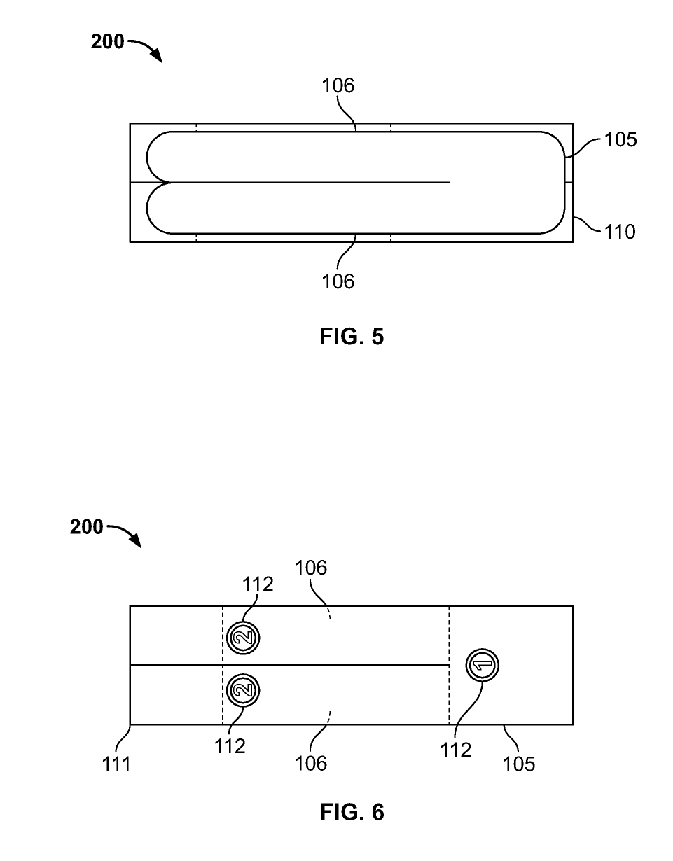

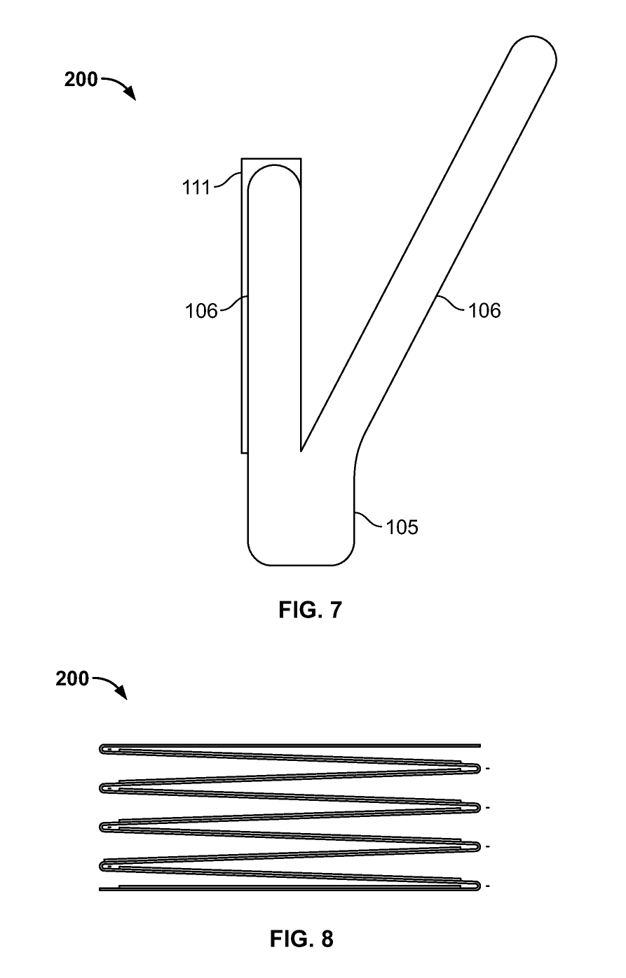

Two embodiments of contusion patch are illustrated in FIGS. 1 to 8. The patches 100, 200 are for use in the treatment of soft tissue injuries such as contusions and minor abrasions. Each patch 100, 200 is easily applied to any minor soft tissue injury irrespective of which body part has sustained the injury. Furthermore, the orientation of the patch 100, 200 on the user's body does not influence its effectiveness, as the patch is not compensating for a musculature imbalance or strain. This universal applicability and uncomplicated application process make the patch 100, 200 suitable for general consumer use.

Each patch 100, 200 is fabricated from a high stretch therapeutic tape 101 with an adhesive backing. The adhesive backing secures the tape 101 to a contusion when the patch 100, 200 is applied to a user's skin. In both illustrated embodiments 100, 200, the tape 101 is less than about 5 inches (13 cm) in length and about 2 inches (5 cm) in width when unstretched. This compact size allows the patch to be used on small injuries (e.g. localized bruises), and gives less opportunity for the exposed tape to stick to itself. This contrasts with more elaborate tapes currently on the market.

The tape 101 is functionally divided into sections. The sections are preferably applied to a user's skin in a prescribed sequence. Each section is made from the same strip of material and shares the same adhesive backing. The functional sections of the illustrated tape 101 include an anchoring portion 105 and a plurality of fingers 106 that extend from the anchoring portion 105. The anchoring portion 105 of the tape 101 is adhesively secured to the skin directly over a contusion. The fingers 106 are adhesively secured to surrounding areas of the user's skin without regard to their orientation on the user's body. The adhesive backing extends uninterrupted across each section of the tape 101.

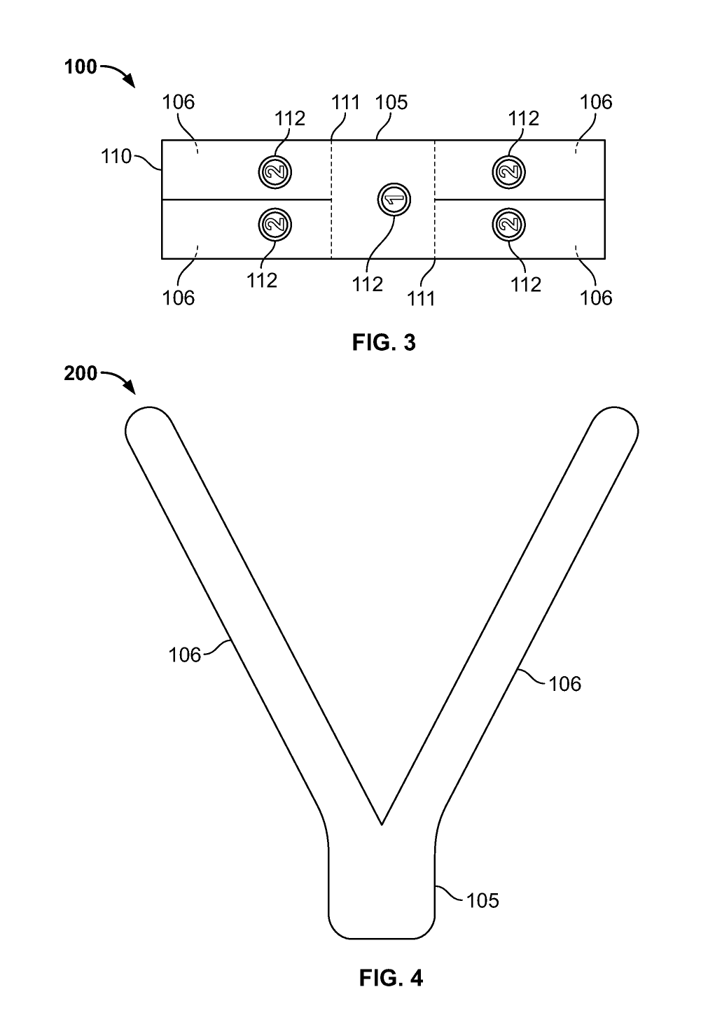

The adhesive backing of the tape 101 is covered with a frangible release liner 110. The release liner 110 is removed before the tape 101 is applied. The release liner 110 reduces contamination of the adhesive backing (preventing dust and other debris from sticking to the adhesive) and inadvertent adhesion during handling. The release liner 110 is scored at the junctions between adjacent sections of the tape 101 (as illustrated in FIGS. 3 and 6). The illustrated release liner 110 has scoring 111 between the anchoring portion 105 and the fingers 106. The scoring 111 assists separation of the release liner 110 covering adjacent sections of the tape 101, allowing portions of the release liner 110 to be selectively removed during application. Ideally the release liner 110 is only removed from a section of the tape 101 when the user is ready to apply the respective section. This prevents the adhesive backing from inadvertently sticking to the user or their clothing.

The release liner 101 has a greater surface area than the corresponding tape 101. A strip 115 of exposed release liner 110 extends beyond the parameter of the tape 101 in the illustrated embodiments. The exposed strip 115 extends around the entire perimeter of the tape 101, creating a boarder that resists edges of the tape 101 inadvertently peeling away from the release liner 110.

The contusion patches may be marked with visual indicators to direct a user when applying the patch 100, 200 to an injury. The markings are ideally applied to the release liner 110. The illustrated contusion patches 100, 200 have numerals 112 printed on the respective release liners 111. The numerals 112 indicate the staggered order in which each section of the tape 101 is to be applied.

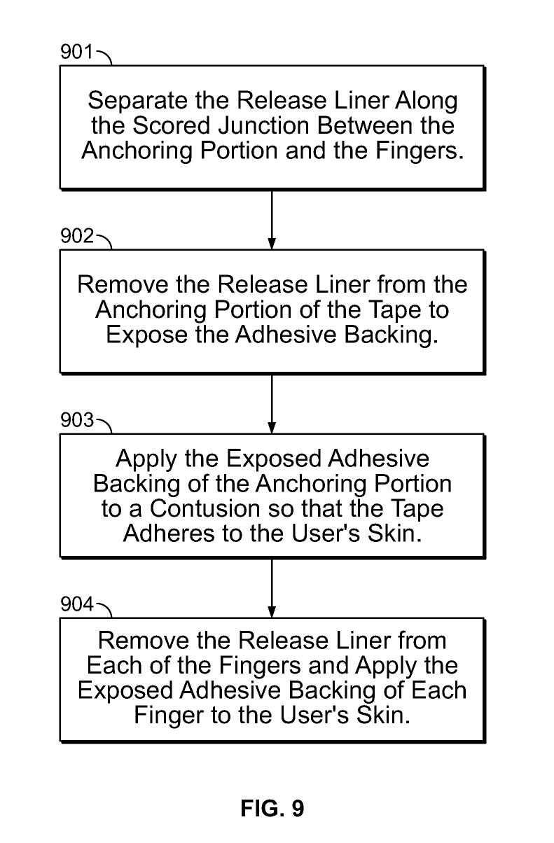

The patches 100, 200 may also be supplied with instructions that assist the user to apply the tape 101. The instructions ideally direct the user to apply the patch 100, 200 to a nonspecific part of the user's body without regard to the orientation of the fingers and may reference visual indicators marked on the tape 101 or release liner 100, 200. An exemplary set of instructions for applying the contusion patch to a user's skin are presented in FIG. 9. The instructions direct the user to:

i. separate the release liner 110 along the scored junction 111 between adjacent sections of the tape 101, such as the junction between the anchoring portion 105 and the fingers 106 (step 901),

ii. remove the release liner 110 from a particular section of the tape 101 to expose the adhesive backing of that section, ideally sections of the release liner 110 are removed in an order identified by marked indicators and the release liner 110 is removed from the anchoring portion 105 of the tape 101 first (step 902),

iii. apply the exposed adhesive backing of the first section (ideally the anchoring portion 105) to a contusion so that the tape 101 adheres to the user's skin (step 903), and

iv. remove the release liner 110 from each of the remaining sections (usually the fingers 106) and apply the exposed adhesive backing of each section/finger 106 in turn to the user's skin (step 904).

The instructions may also direct the user to tension sections of the tape 101 during application. The instructions may direct the user to stretch one or more of the fingers 106 after the release liner 110 has been removed and to secure the respective finger 106 to the user's skin while stretched. This retains some tension in finger 106 following adhesion to the user's skin. The residual tension pulls the anchoring portion 105 toward the finger 106, lifting the skin over the contusion. The user may be instructed to secure the anchoring portion 105 of the tape 101 without stretching, so that there is no pre-tensioning in the anchoring portion 105.

Each section of the tape 101 is secured in place by pressing the exposed adhesive backing against the user's skin. Ideally, the release liner 110 is only removed from each section when the user is ready to apply the respective section. For instance, the anchoring portion 105 may be secured to the contusion before the release liner 110 is removed from any of the fingers 106. The release liner 110 is ideally removed from each finger 106 in turn, so that the adhesive backing is only exposed immediately prior to application.

The tape 101 is preferably breathable. The illustrated tape 101 has a woven fabric base that allows liquid to pass through gaps between adjacent threads in the weave. One side of the fabric is coated with a skin-safe high tack adhesive (the adhesive backing) that holds the tape 101 in contact with the skin. The coating is ideally a high strength dermatologically-acceptable acrylic adhesive.

The underlying fabric weave of the tape 101 produces desirable anisotropic deformation characteristics when the tape 101 is stretched. The tape 101 exhibits a high stretch ratio when tensioned coaxially with the fingers 106 (as illustrated in FIG. 8). Ideally the stretch ratio of the tape 101 when extended coaxially with the fingers 106 roughly equates to the elasticity of human skin. This corresponds to a stretch ratio of about 1.4. The stretch ratio may be higher without adversely affecting the tapes performance. The tape 101 is significantly more resistant to deformation transverse to the direction the fingers 106 extend. This resistance to transverse deformation maintains the tape 101 width when the fingers 106 are stretched, preventing significant inward contraction. It also opposes transverse extension of the tape if tension is applied transverse to the fingers 101.

An ‘X’ configuration patch 100 is illustrated in FIGS. 1 to 3. The ‘X’ configuration patch 100 has four fingers 106 that extend from the central anchoring portion 105. The fingers 106 are arranged in pairs that extend from opposing sides of the anchoring portion 105. An alternate ‘Y’ configuration patch 200 is illustrated in FIGS. 4 to 6. The ‘Y’ configuration patch 200 has two fingers 106 that both extend from the same side of the anchoring portion 105. Both tape configurations can be applied without concern for orientation so long as the anchoring portion is secured to the skin over the contusion.

The illustrated contusion patches may be bundled together in packs or kits containing multiple patches. The patches are ideally connected end-to-end and stacked flat within the desired packaging to reduce the overall size of the kit. This stacking arrangement is illustrated in FIG. 8. Adjacent patches in the kit are connected by the frangible release 110 liner. The patches are folded one atop the other, with each successive patch resting on top of a previous patch as illustrated. The frangible release liner is scored between adjacent patches (similarly to the scoring between adjacent sections of each patch) in the kit to facilitate separation of adjacent patches.

While the foregoing is directed to embodiments of the invention, other and further embodiments of the invention may be devised without departing from the basic scope thereof, and the scope thereof is determined by the claims that follow.

1. A contusion patch kit, comprising:
a high stretch therapeutic tape with an adhesive backing, the tape having an anchoring portion that adheres to a contusion and a plurality of fingers that extend from the anchoring portion,
a frangible release liner covering the adhesive backing of the tape, the release liner having a greater surface area than the tape so that an exposed strip of the release liner extends around the tape's perimeter, the release liner being scored at junctions between the anchoring portion and the fingers to allow selective removal of the release liner during staggered application of the tape, and
instructions directing a user to apply the contusion patch to a nonspecific part of the user's body using any orientation of the fingers in the user's discretion.

2. The kit of claim 1, wherein the therapeutic tape is less than about 5 inches in length and about 2 inches in width when unstretched.

3. The kit of claim 1, wherein the therapeutic tape has a stretch ratio of about 1.4 or greater.

4. The kit of claim 1, wherein the release liner is marked with numerals that indicate the order in which sections of the patch are to be applied.

5. The kit of claim 1, wherein the tape has four fingers that extend from the anchoring portion, the fingers being arranged in pairs that extend from opposing sides of the anchoring portion to allow the tape to be applied in an ‘X’ configuration.

6. The kit of claim 1, wherein the tape has two fingers that extend from one side of the anchoring portion to allow the tape to be applied in a ‘Y’ configuration.

7. The kit of claim 1, wherein the patch is supplied in a kit with instructions for applying the patch to a contusion, the instructions directing a user to:
i. separate the release liner along the scored junction between the anchoring portion and the fingers,
ii. remove the release liner from the anchoring portion of the tape to expose the adhesive backing,
iii. apply the exposed adhesive backing of the anchoring portion to a contusion so that the tape adheres to the user's skin, and
iv. remove the release liner from each of the fingers and apply the exposed adhesive backing of each finger to the user's skin.

8. The kit of claim 7, wherein the instructions direct the user to stretch each finger after removing the release liner and secure the respective finger in place while stretched by pressing the exposed adhesive backing against the user's skin.

9. The kit of claim 7, wherein the instructions direct the user to secure the anchoring portion of the tape to the user's skin without any tension in the anchoring portion.

10. The kit of claim 7, wherein the kit includes a plurality of contusion patches connected end-to-end, the patches being folded flat with each successive patch resting on top of a previous patch, adjacent patches being connected by the frangible release liner.

11. The kit of claim 10, wherein the frangible release liner is scored between adjacent patches.

12. The kit of claim 1, wherein the therapeutic tape is coated with a dermatologically-acceptable acrylic adhesive to create the adhesive backing.

13. A contusion patch kit, comprising:
a high stretch therapeutic tape with an adhesive backing, the tape being functionally divided into sections including an anchoring portion that adheres to a contusion and a plurality of fingers that extend from the anchoring portion,
a frangible release liner covering the adhesive backing of the tape, the release liner having a greater surface area than the tape so that an exposed strip of the release liner extends around the tape's perimeter, the release liner being scored at junctions between adjacent sections of the tape to allow selective removal of the release liner during staggered application of the tape, the release liner being marked with indicators that dictate the order in which each section of the tape is to be applied, and
instructions for applying the contusion patch to a nonspecific part of the user's body using any orientation of the fingers in the user's discretion, the instructions referencing the release liner indicators and directing a user to:
i. separate the release liner along the scored junction between adjacent sections of the tape,
ii. remove the release liner from a particular section of the tape indentified by one of the indicators to expose the adhesive backing of that section,
iii. apply the exposed adhesive backing of the section to a contusion so that the tape adheres to the user's skin, and
iv. remove the release liner from each of the other sections in an order dictated by the indicators marked on the release liner and apply the exposed adhesive backing of each section in turn to the user's skin.

14. The kit of claim 13, wherein the tape is less than about 5 inches in length and about 2 inches in width when unstretched

15. The kit of claim 13, wherein the instructions direct the user to secure the tape to the contusion without any tension in the tape.

16. The kit of claim 13, wherein the therapeutic tape has a stretch ratio of about 1.4 or greater.

17. The kit of claim 13, wherein the indicators marked on the release liner are numerals that indicate the order in which sections of the patch are to be applied.

18. The kit of claim 13, wherein the kit includes a plurality of contusion patches connected end-to-end, the patches being folded flat with each successive patch resting on top of a previous patch, adjacent patches being connected by the frangible release liner.

19. The kit of claim 18, wherein the frangible release liner is scored between adjacent patches.
